(19) United States
(12) Patent Application Publication
(10) Pub. No.: US 2012/0101159 A1
(43) Pub. Date: Apr. 26, 2012

(54) IBUPROFEN LYSINATE ORAL SUSPENSION

(75) Inventor: Maite Tarre Perez, Barcelona (ES)

(73) Assignee: Laboratorio De Aplicaciones Farmacodinamicas, S.A., Barcelona (ES)

(21) Appl. No.: 13/266,730

(22) PCT Filed: Apr. 27, 2010
(86) PCT No.: PCT/ES10/00185

§ 371 Date: Jan. 5, 2012

(30) Foreign Application Priority Data
Apr. 27, 2009 (EP) 09380086.0
Apr. 27, 2009 (ES) P200901093

(51) Int. Cl. A61K 31/205; A61P 29/00

(52) U.S. Cl. 514/555

(57) ABSTRACT

The present invention refers to an ibuprofen lysinate-based pharmaceutical composition in the form of oral suspension and to the preparation procedure thereof.

### FIELD OF THE INVENTION

The present invention refers to an ibuprofen lysinate-based pharmaceutical composition as oral suspension and to the preparation procedure thereof.

### BACKGROUND OF THE INVENTION

Fever and pain are symptoms of several childhood illnesses, which occur as a result of an alteration in the body, an infection and other causes.

Nowadays, there exist different drugs in the pharmaceutical market for treating these symptoms, most of which are ibuprofen- and paracetamol-based drugs. Ibuprofen therapeutic void is well-known between the drug administration and the beginning of the antipyretic and analgesic effect, being an objectionable limitation in the treatment of fever and pain and in the time that goes by between the drug administration and the decrease in body temperature and pain relief. Analgesics, antipyretics and anti-inflammatory drugs are not water soluble like ibuprofen; they leave certain areas of the gastric mucosa exposed for a long time to the histolesive action of high concentrations of the active principle.

Ibuprofen lysinate is a salt obtained through a process of chemical synthesis, from ibuprofen and lysine amino acid. One of the most important physicochemical transformations underwent by ibuprofen when saltified with lysine is that it becomes a substance with little solubility in a watery solution since it is quickly and completely water-soluble. This means a more rapid and homogeneous gastrointestinal absorption which transforms the pharmacokinetic properties of ibuprofen and adds differential and advantageous therapeutic characteristics, although its organoleptic taste characteristics become worse.

One of the main advantages of ibuprofen lysinate is that, being an orally administered soluble salt, it homogeneously disperses over a wider gastric surface, enabling, on the one hand, a more rapid and homogeneous absorption, and on the other hand, a noticeably smaller incidence of side effects associated to analgesics, antipyretics and anti-inflammatory drugs which are not water soluble. These advantages translate into a better kinetic and tolerance profile for ibuprofen lysinate compared to ibuprofen, and consequently, into better clinical results since they allow a faster therapeutic action, which is a critical property for treating fever and pain, and a less harmful effect on gastric mucosa, which is a differential and advantageous difference compared to ibuprofen. The combination of lysinate ibuprofen and beta-cyclodextrins improves these properties and solves the problem of palatability producing an acceptable administration.

EP1129709 refers to an ibuprofen-based pharmaceutical composition in the form of powder, whose active component is ibuprofen lysinate combined with beta-cyclodextrins. This patent presents the advantages described of ibuprofen lysinate but excludes its administration to the pediatric population, since drug dosage for this population is carried out according to their body weight and, in oral administration the only pharmaceutical forms which enable to administer a variable dose are solutions or suspensions.

In the market there exist different ibuprofen suspensions; however, none of them contains as active component ibuprofen lysinate, which is a different active principle from ibuprofen due to its significant differences as regards safety and efficacy, and they normally contain sugar, excluding their administration to patients suffering from diabetes.

There is then a need to find a way to orally administer ibuprofen lysinate to the pediatric population so that this population can benefit from the described therapeutic advantages of this active component compared to analgesics, antipyretics and anti-inflammatory drugs which are not water soluble.

### DESCRIPTION OF THE INVENTION

The present invention refers to an ibuprofen lysinate-based pharmaceutical composition in the form of oral suspension, with analgesic, antipyretic and anti-inflammatory activity, not containing contain sucrose, having a variable dosage and being adaptable to the patient's weight so that it is intended to the pediatric population and suitable for patients suffering from diabetes or fructose/sucrose intolerance.

Thus, a first aspect of the present invention refers to a pharmaceutical composition containing ibuprofen lysinate and pharmaceutically acceptable excipients in the form of oral suspension.

In a more specific aspect, the pharmaceutical composition contains ibuprofen lysinate and pharmaceutically acceptable excipients in the form of oral suspension and it does not contain sucrose.

In a more specific aspect, the ibuprofen lysinate is combined with cyclodextrins, in a more specific embodiment, the cyclodextrins are beta-cyclodextrins, and in another specific embodiment, the cyclodextrins are hydroxypropyl beta-cyclodextrins.

In a more specific aspect, the ibuprofen lysinate is combined with beta-cyclodextrin with a weight ratio of ibuprofen lysinate/beta-cyclodextrin comprised between 1:1 and 1:5.

In the present invention by “ibuprofen lysinate combined with beta-cyclodextrin” we refer to ibuprofen lysinate encapsulation in the beta-cyclodextrins.

In another more specific aspect, the pharmaceutically acceptable excipients contained in the pharmaceutical composition of the invention, are selected from the group formed by colloidal agents, preservative agents, diluting agents, sweetening agents, aromatic agents and artificial colors.

In another more specific aspect, the colloidal agent is comprised between 0.4 and 3% in weight. In another more specific aspect, the colloidal agent is microcrystalline cellulose combined with sodium carboxymethylcellulose.

In another more specific aspect, the preservative agents of the pharmaceutical composition of the present invention are comprised between 0.02 and 2% in weight. In another more specific aspect, the preservative agents are selected among preservatives like paraben or potassium sorbate. In another more specific aspect, the preservative agents are combinations of methylparaben, ethylparaben, propylparaben and potassium sorbate.

In another more specific aspect, the sweetening agent of the pharmaceutical composition of the present invention is comprised between 0.10 and 0.20% in weight; in another more specific aspect, the sweetening agent is sodium sucrose, sodium cyclamate and aspartame.

In another more specific aspect, the pharmaceutical composition of the present invention contains fruit of the forest as aromatic agent.

In another more specific aspect, the pharmaceutical composition of the present invention contains allura red AC as artificial color.

A second aspect of the present invention refers to a procedure for preparing the pharmaceutical composition of the present invention which comprises the following stages:
a) encapsulation of ibuprofen lysinate in cyclodextrins. In a more specific aspect, the cyclodextrins are beta-cyclodextrins. In another more specific aspect, the encapsulation of ibuprofen lysinate is carried out through sifting and ultrarapid mixing of ibuprofen lysinate and beta-cyclodextrins in a 1:1-1:5 ratio during 1 to 20 minutes,
b) solubilization of preservatives in propylene glycol or alternatively, in purified water. In a more specific aspect, the preservatives are selected among preservatives like paraben and potassium sorbate. In a more specific aspect, the solubilization is carried out at a temperature between 60-100° C.,
c) refrigeration of the mix of preservatives in purified water up to a temperature between 25-37° C.,
d) addition into stage c) of the microcrystalline cellulose and sodium carboxymethylcellulose and mixing at high speed during 15 to 60 minutes to form the suspension,
e) addition into stage d) of the diluting agents, sweetening agents, ibuprofen lysinate-beta-cyclodextrin, aromatic agents, artificial colors and purified water qsp. In a more specific aspect, the diluting agents are maltitol and sorbitol, in another more specific aspect, the sweetening agent is sodium sucrose, sodium cyclamate or aspartame; in another more specific aspect, the aromatic agent is fruit of the forest; in another more specific aspect, the artificial color is Allura red,
f) filtration

### DETAILED DESCRIPTION OF THE INVENTION

Ibuprofen lysinate pharmaceutical composition combined with cyclodextrins (Table 1)

TABLE 1

| Pharmaceutical composition | Porcentaje en peso |
|---|---|
| Ibuprofen lysinate | 2-5 |
| Cyclodextrin | 2-20 |
| Colloidal agent | 0.4-3 |
| Preservative agents | 0.02-2 |
| Diluting agents | 2-20 |
| Sweetening agents | 0.10-0.20 |
| Aromatic agents | 0.10-0.50 |
| Artificial colors | 0.006-0.009 |
| Purified water q.s.p. | 100 ml |

### Example 1

### Ibuprofen Lysinate Pharmaceutical Composition Combined with Beta-Cyclodextrins (Table 2)

TABLE 2

| Pharmaceutical composition | Porcentaje en peso |
|---|---|
| Ibuprofen lysinate | 2-5 |
| Beta-cyclodextrin | 2-20 |
| Microcrystalline cellulose/sodium | 0.4-3 |
| carboxymethylcellulose | |
| Methylparaben, propylparaben, ethylparaben | 0.1-0.5 |
| Maltitol | 5.00-20.00 |
| Sorbitol | 2.00-3.50 |
| Sodium sucrose | 0.10-0.20 |
| Fruit of the forest aromatic agent | 0.10-0.50 |
| Allura red AC | 0.006-0.009 |
| Purified water q.s. p. | 100 ml |

### Example 2

### Ibuprofen Lysinate Pharmaceutical Composition Combined with Cyclodextrins (Table 3)

TABLE 3

| Pharmaceutical composition | Porcentaje en peso |
|---|---|
| Ibuprofen lysinate | 2 |
| Beta-cyclodextrin | 6.5 |
| Microcrystalline cellulose/sodium | 1 |
| carboxymethylcellulose | |
| Methylparaben, propylparaben, ethylparaben | 0.2 |
| Maltitol | 12 |
| Sorbitol | 2 |
| Sodium sucrose | 0.12 |
| Fruit of the forest aromatic agent | 0.15 |
| Allura red AC | 0.006 |
| Purified water q.s.p. | 100 ml |

### Example 3

### Ibuprofen Lysinate Pharmaceutical Composition Combined with Cyclodextrins (Table 4)

TABLE 4

| Pharmaceutical composition | Porcentaje en peso |
|---|---|
| Ibuprofen lysinate | 4 |
| Beta-cyclodextrin | 16 |
| Microcrystalline cellulose/sodium | 0.6 |
| carboxymethylcellulose | |
| Propylparaben | 0.04 |
| Potassium sorbate | 1.5 |
| Propylene glycol | 2 |
| Maltitol | 9 |
| Sorbitol | 3.5 |
| Aspartame | 0.15 |
| Fruit of the forest aromatic agent | 0.2 |
| Allura red AC | 0.007 |
| Purified water q.s.p. | 100 ml |

### Example 4

### Ibuprofen Lysinate Pharmaceutical Composition Combined with Beta-Cyclodextrins (Table 5)

TABLE 5

| Pharmaceutical composition | Porcentaje en peso |
|---|---|
| Ibuprofen lysinate | 3.5 |
| Beta-cyclodextrin | 10 |
| Microcrystalline cellulose/sodium | 0.8 |
| carboxymethylcellulose | |
| Methylparaben, propylparaben, ethylparaben | 0.25 |
| Potassium sorbate | 1 |
| Propylene glycol | 3 |
| Maltitol | 9 |
| Sorbitol | 2 |
| Sodium sucrose | 0.20 |
| Fruit of the forest aromatic agent | 0.14 |
| Allura red AC | 0.007 |
| Purified water q.s.p | 100 ml |

### Example 5

### Method for Preparing Ibuprofen Lysinate Pharmaceutical Composition

The procedure for preparing the ibuprofen lysinate pharmaceutical composition of the present invention was carried out according to the following steps:

The ibuprofen lysinate is encapsulated through sifting and ultrarapid mixing of ibuprofen lysinate and cyclodextrins in a 1:1-1:5 ratio during 1 to 20 minutes. Next, there is a solubilization of preservatives in purified water at a temperature between 60-100° C.; said solubilization can be made in propylene glycol. Said mix (preservatives in purified water) was refrigerated until up to a temperature between 25-27° C. Later, the colloidal agent is added to said mix and mixed at high speed during 15 to 60 minutes, until the suspension was formed. The diluting agents, the sweetening agent, the ibuprofen lysinate-cyclodextrin, the aromatic agent, the artificial color and purified water qsp were added to said mix and all components were mixed. Finally, the mix was filtered.

### Example 6

### Procedure for Preparing the Ibuprofen Lysinate Pharmaceutical Composition Described in Example 2.

a) encapsulation of ibuprofen lysinate through sifting and ultrarapid mixing of ibuprofen lysinate and beta-cyclodextrins in a 1:1-1:5 ratio during 1 to 20 minutes.
b) solubilization of methylparaben, ethylparaben and propylparaben in purified water at a temperature between 60-100° C.
c) refrigeration of the mix of preservatives in purified water up to a temperature between 25-37° C.
d) addition into stage c) of the microcrystalline cellulose and sodium carboxymethylcellulose and mixing at high speed during 15 to 60 minutes to form the suspension.
e) addition into stage d) of the following compounds:
   maltitol
   sorbitol
   sodium sucrose
   fruit of the forest aromatic agent
   allura red AC
   purified water qsp
f) filtration

### Example 7

### Procedure for Preparing the Ibuprofen Lysinate Pharmaceutical Composition Described in Example 3.

a) encapsulation of ibuprofen lysinate through sifting and ultrarapid mixing of ibuprofen lysinate and cyclodextrins in a 1:1-1:5 ratio during 1 to 20 minutes.
b) solubilization of propylparaben and potassium sorbate in propylene glycol.
c) addition into stage b) of the microcrystalline cellulose and sodium carboxymethylcellulose and mixing at high speed during 15 to 60 minutes to form the suspension.
d) addition into stage c) of the following compounds:
   maltitol
   sorbitol
   aspartame
   fruit of the forest aromatic agent
   allura red AC
   purified water qsp
f) filtration

### Example 8

### Procedure for Preparing the Ibuprofen Lysinate Pharmaceutical Composition Described in Example 4.

a) encapsulation of ibuprofen lysinate through sifting and ultrarapid mixing of ibuprofen lysinate and cyclodextrins in a 1:1-1:5 ratio during 1 to 20 minutes.
b) solubilization of methylparaben, propylparaben, ethylparaben and potassium sorbate in propylene glycol.
c) addition into stage b) of the microcrystalline cellulose and sodium carboxymethylcellulose and mixing at high speed during 15 to 60 minutes to form the suspension.
d) addition into stage c) of the following compounds:
   maltitol
   sorbitol
   sodium sucrose
   fruit of the forest aroma
   allura red AC
   purified water qsp
f) filtration

1. Pharmaceutical composition containing ibuprofen lysinate, the group formed by colloidal agents, and pharmaceutically acceptable excipients in the form of oral suspension.

2. Pharmaceutical composition according to claim 1, wherein said colloidal agents are comprised between 0.4 and 3% by weight.

3. Pharmaceutical composition according to claim 1 or 2, wherein said colloidal agents are microcrystalline cellulose in combination with with sodium carboxymethylcellulose.

4. Pharmaceutical composition according to any of claims 1 to 3, not containing sucrose.

5. Pharmaceutical composition according to any of claims 1 to 4, wherein the ibuprofen lysinate is combined with cyclodextrins.

6. Pharmaceutical composition according to claim 5, wherein the weight ratio between ibuprofen lysinate and said cyclodextrins is comprised between 1:1 and 1:5.

7. Pharmaceutical composition according to any of claim 5 or 6, wherein said cyclodextrins are beta-cyclodextrins or hydroxypropyl beta-cyclodextrins.

8. Pharmaceutical composition according to any of the preceding claims, wherein the pharmaceutically acceptable excipients are selected from the group formed by preservative agents, maltitol and sorbitol as diluting agents, sweetening agents, aromatic agents, and artificial colors.

9. Pharmaceutical composition according to claim 8, wherein said preservative agent is selected among parabens and potassium sorbate.

10. Pharmaceutical composition according to claim 9, wherein said parabens are methylparaben, ethylparaben and/or propylparaben.

11. Pharmaceutical composition according to any of claims 8 to 10, wherein said preservative agent is comprised between 0.02 and 2% in weight.

12. Pharmaceutical composition according to claim 8, wherein said diluting agents are comprised between 2 and 20% in weight.

13. Procedure for preparing a pharmaceutical composition according to any of the preceding claims, characterized in that it comprises the following stages:
a) encapsulation of ibuprofen lysinate in the cyclodextrins,
b) solubilization of the preservative in purified water or in propylene glycol,
c) addition into stage b) of the colloidal agent and mixing at high speed to form the suspension,
d) addition into stage c) of the diluting agents, the sweetening agents, the ibuprofen lysinate-cyclodextrin, the aromatic agent, the artificial color and purified water qsp., and
e) filtration.

14. Procedure according to claim 13, wherein the stage a) is carried out by sieving and ultrarapid mixing of ibuprofen lysinate and beta-cyclodextrins in a 1:1 to 1:5 weight ratio.
